(19) United States
(12) Patent Application Publication
(10) Pub. No.: US 2016/0220670 A1
(43) Pub. Date: Aug. 4, 2016

(54) BOOSTING THE EFFICACY OF DNA-BASED VACCINES WITH NON-THERMAL DBD PLASMA

(71) Applicant: EP Technologies LLC, Akron, OH (US)

(72) Inventors: Sameer Kalghatgi, Copley, OH (US); Daphne Pappas Antonakas, Hudson, OH (US); Tsung-Chan Tsai, Cuyahoga Falls, OH (US); Robert L. Gray, Hudson, OH (US); Sarah D. Stone, Akron, OH (US)

(21) Appl. No.: 15/012,304

(22) Filed: Feb. 1, 2016

Related U.S. Application Data
(63) Continuation-in-part of application No. 14/560,343, filed on Dec. 4, 2014.
Application No. 14/560,343 is a continuation-in-part of application No. 14/610,467, filed on Jan. 30, 2015.
(60) Provisional application No. 61/911,536, filed on Dec. 4, 2013.
(60) Provisional application No. 61/933,384, filed on Jan. 30, 2014.

(51) Int. Cl. A61K 41/00; A61N 5/06; A61N 1/44; A61K 39/00; A61M 37/00

(52) U.S. Cl. CPC A61K 41/00; A61K 39/00; A61M 37/00; A61N 1/44; A61N 5/0616; A61K 2039/53; A61M 2202/30; A61M 2037/0007

(57) ABSTRACT

The efficacy of a DNA-based vaccine in terms of eliciting a desired immune response is enhanced by directing a non-thermal plasma generated by a non-thermal plasma generator at the site on the patient's skin where the vaccine was previously introduced.

### CROSS-REFERENCE TO RELATED APPLICATIONS

This application is a continuation-in-part of U.S. application Ser. No. 14/560,343, filed Dec. 4, 2014, and published as US 2015/0151135 on Jun. 4, 2015 (Atty. Docket No. 35416/04028), which claims priority to U.S. provisional application Ser. No. 61/911,536, filed Dec. 4, 2013 (Atty. Docket No. 35416/04012). In addition, this application is also a continuation-in-part of U.S. application Ser. No. 14/610,467, filed Jan. 30, 2015, and published as US 2015/0209505 on Jul. 30, 2015 (Atty. Docket No. 35416/04040), which claims priority to U.S. provisional application Ser. No. 61/933,384, filed Jan. 30, 2014 (Atty. Docket No. 35416/04013). The disclosures of each of these applications are incorporated herein by reference in their entireties.

### BACKGROUND

In order for a DNA-based vaccine to be immunogenic (i.e., effective in the sense of conferring protection against a disease), two basic steps are necessary. In the first step, the vaccine must be delivered to a site inside the skin or muscle which is at or in close proximity to target cells that are capable of taking up the DNA-based vaccine and expressing proteins which generate the desired immune response such as, for example, skin cells in the epidermis, antigen presenting cells like Langerhans cells, dendritic cells, macrophages, dermal dendritic cells, CD4+ T cells, CD8+ T cells etc., that reside in the skin, and so forth. In the second step, the vaccine must be delivered to the interior of the above mentioned target cells by passing through their bilayer cell membrane. The primary focus of this invention is on the second of these steps.

In this regard, it is already known that the efficacy of previously-injected DNA-based vaccines in terms of eliciting a desired immune response can be enhanced by applying a certain type of plasma to the injection site. See, Richard J. Connolly, Plasma Mediated Molecular Delivery, PhD Thesis, University of South Florida, 2010. See, also, Connolly et. al, Non-contact helium-based plasma for delivery of DNA-based vaccines—Enhancement of humoral and cellular immune responses, Human Vaccines & Immunotherapeutics 8:11, 1729-1733, November © 2012, Landes Bioscience.

In the processes described there, an atmospheric pressure DC plasma jet that is generated by applying a continuously operating, high voltage, direct electrical current to a flowing column of helium gas is directed to the site of the previous DNA injection.

Meanwhile, U.S. 2014/0188071 to Jacofsky et al. describes another process for using a non-thermal plasma to enhance the efficacy of a previously-applied medication or other substance. In this process, a non-thermal plasma generated by a dielectric barrier discharge (DBD) plasma generator is used for this purpose. However, an important feature of this process is that its non-thermal plasma is applied so that it “passes through” the medicine or substance before being applied to the substrate. In other words, the non-thermal plasma directly contacts the substance being applied. As a result, this approach cannot be used on medications which have been previously injected, since they are no longer exposed for direct contact by the plasma. More importantly, using this technology on DNA-based vaccines poses a substantial risk that they would be rendered ineffective, since it well known that non-thermal plasmas will readily oxidize many drugs, substances and materials and modify them irreversibly and in a detrimental manner.

### SUMMARY

In accordance with this invention, we have found that non-thermal plasmas can be used to enhance the efficacy of previously-injected DNA vaccines in terms of eliciting a desired immune response in a manner which avoids the shortcomings mentioned above by using a dielectric barrier discharge (“DBD”) plasma generator as the source for these non-thermal plasmas.

Thus, this invention provides a process for enhancing the efficacy of a DNA vaccine in connection with eliciting a desired immune response in which the DNA vaccine has been previously introduced into the body of a patient, the process comprising directing a non-thermal plasma generated by a DBD plasma generator at the application site where the DNA vaccine was introduced.

In addition, this invention also provides an improved process for treating a patient with a DNA vaccine to accomplish a desired immune response, this process comprising injecting the patient with a DNA vaccine capable of achieving a desired immune response and thereafter applying a non-thermal plasma generated by a DBD plasma generator to the injection site where this DNA vaccine was injected.

In still another embodiment, this invention also provides an improved process for treating a patient with a DNA-based vaccine to accomplish a desired immune response, this process comprising applying a first non-thermal plasma to the application site where this DNA-based vaccine will be applied, topically applying a DNA-based vaccine capable of achieving the desired immune response to this application site, allowing this DNA-based vaccine to migrate to inside the skin of the patient and thereafter directing a second non-thermal plasma at the application site where the DNA-based vaccine was injected, wherein both the first and second non-thermal plasmas are generated by DBD plasma generators.

### BRIEF DESCRIPTION OF THE DRAWINGS

This invention may be more readily understood by reference to the following drawings in which:

FIG. 1 is a simplified schematic diagram illustrating the structure of a DC plasma generator of the type used in certain prior-art work mentioned above; and

FIG. 2a is a simplified schematic diagram similar to FIG. 1 illustrating the structure of a jet-type DBD plasma generator of the type that can be used in this invention; and

FIG. 2b illustrates the structure of a jet-type DBD plasma generator similar to that of FIG. 2a, except that the plasma generator of FIG. 2b includes an additional grounded counter electrode; and

FIGS. 3a, 3b and 3c are diagrams of the equivalent electrical circuits which illustrate the operation of the DC plasma generator of FIG. 1 and the DBD plasma generator of FIG. 2; and

FIG. 4a is a waveform diagram which illustrate the electrical current generated during operation of the plasma and the corresponding equivalent electrical circuits of FIG. 3a (DC plasma generator), while FIG. 4b is a combination waveform diagram which illustrates both the voltage applied and the electrical current generated during operation of the plasma and the corresponding equivalent electrical circuits FIG. 3b (DBD plasma generator); and

FIG. 5 illustrates a jet-type DBD plasma generator similar to the jet-type DBD non-thermal plasma generators of FIGS. 2a and 2b that can be used to generate an indirect non-thermal plasma for use in the inventive process, and

FIGS. 6 and 7 illustrate large area type DBD non-thermal plasma generators that can be used for generating direct and indirect non-thermal plasmas for use in the inventive process, and

FIG. 8a illustrates a typical voltage waveform diagram produced when a high voltage power source is operated to generate high voltage pulses having microsecond pulse widths; FIG. 8b illustrates the associated current waveform that is produced when the power source of FIG. 8a is powering a DBD plasma generator of the type used in this invention; and FIG. 8c illustrates what we mean by “duty cycle” in connection with a power source of the type illustrated in FIG. 8a; and

FIGS. 9a and 9b are waveform diagrams similar to those of FIGS. 8a and 8b which illustrate the voltage and corresponding current waveforms that are produced and generated when a power source is operated to produce high voltage pulses having nanosecond pulse widths; and

FIG. 10 illustrates a simple alternating voltage and current waveform that can be generated by a power source for driving the operation of DBD plasma generator in accordance with yet another embodiment of this invention; and

FIGS. 11a and 11b are block diagrams which illustrate two examples of vaccination protocols involving multiple vaccinations which can be carried out in accordance with this invention; and

FIGS. 12a-12f are schematic diagrams which illustrate a number of additional vaccination protocols that can be carried out in accordance with this invention; and

FIGS. 13a and 13b are block diagrams similar to FIGS. 11a and 11b which illustrate two additional vaccination protocols that be carried out in accordance with this invention when the DNA-based vaccine is delivered from outside the patient's body to inside the patient's body by topical application; and

FIGS. 14 and 15 are graphical representations of the results obtained in the following Examples 1 and 2 of this disclosure, respectively.

### DETAILED DESCRIPTION

### Plasma

Plasma is the fourth state of matter. A plasma is an energized gas which is either fully ionized or partially ionized, having one or more electrons that are not bound to an atom or molecule. Plasmas, which are fully ionized, are known as “thermal” or “hot” plasmas, because they exist at very high temperatures. In such plasmas, the electrons, ions and neutrals are said to be in thermal equilibrium with one another in the sense that they exist at essentially the same high temperature. Plasmas, which are only partially ionized, are known as “non-thermal” or “non-equilibrium” or “cold” plasmas. In these plasmas, only a small percentage of the atoms or molecules are ionized. As a result, the heavier particles including ions and neutrals are at a much lower temperature than the electrons. The overall result is that the gas temperature of the non-thermal plasma, as a whole, is often at or near room temperature. Nonetheless, such non-thermal plasmas still generate strong electric fields and contain high concentrations of energetic and chemically active species like reactive oxygen and nitrogen species (RONS).

As is well understood in the art, two different types of plasma generators can be used to generate non-thermal plasmas. In one type, which we refer to as a “DC” or “non-DBD” plasma generator, the gas to be converted into a plasma is in direct contact with the electrodes of the device. In addition, a continuously operating direct electrical current is used to establish and maintain a constant large applied voltage, e.g., 1 to 40 kV, between these electrodes.

In the other type, which is known as a dielectric barrier discharge or “DBD” plasma generator, the gas to be converted into a plasma is electrically insulated from the high voltage electrode of the device by an insulating material having a high dielectric constant. In addition, a large applied voltage between this high voltage electrode and ground is supplied in the form of discrete, fast-rising, multiple high voltage pulses with a pulse duration ranging from nanosecond to microseconds, voltages ranging from 1 kV to 40 kV and a pulse repetition frequency ranging from 1 Hz to few tens of kHz. Alternatively, this large applied voltage can be generated in the form of a simple sinusoidal wave continuously alternating between positive and negative voltage maximum ranging from 1 kV to 40 kV at frequencies ranging between 10 Hz and 30 kHz

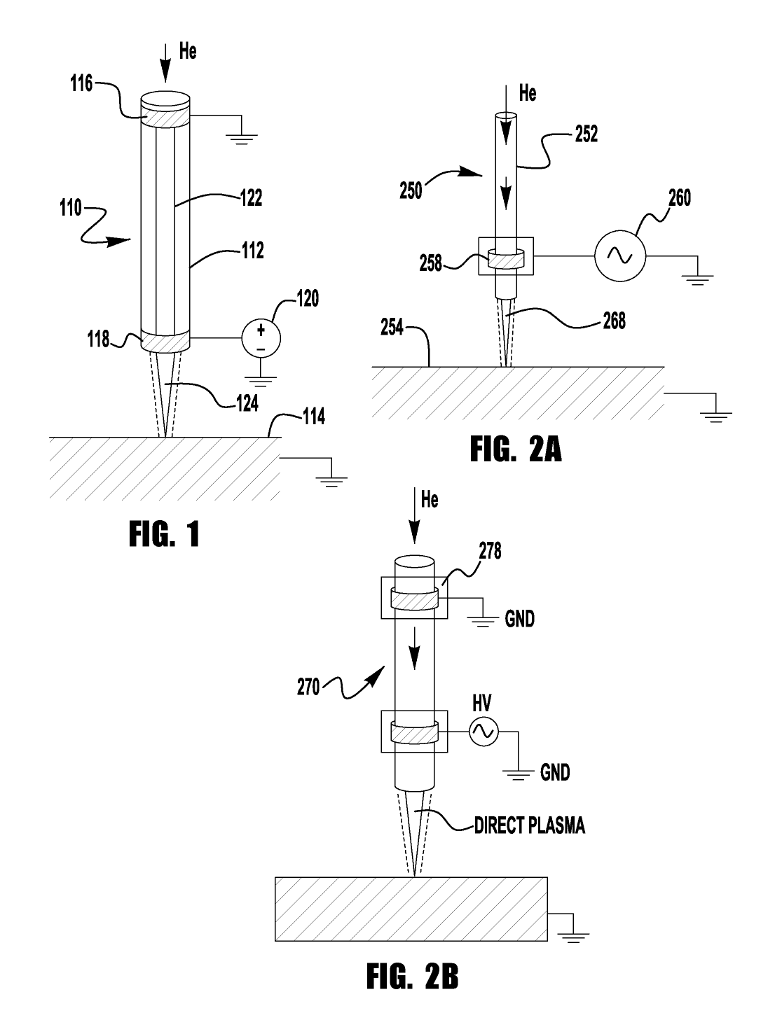

The difference between a DC plasma generator, on the one hand, and a DBD plasma generator, on the other hand, can be more easily understood by reference to FIGS. 1 and 2. FIG. 1 is a simplified schematic diagram illustrating the structure of a jet-type DC plasma generator of the type used in certain earlier work mentioned above, while FIG. 2 is a simplified schematic diagram illustrating the structure of a jet-type DBD plasma generator of the type that can be used in this invention.

As shown in FIG. 1, DC plasma generator 110 of this figure includes a source of noble gas such as helium (not shown), which is provided for directing a jet of this gas through Teflon tube 112 and onto the surface of target 114 to be treated, which is connected to ground. Ambient air, whether at atmospheric or higher pressure, cannot be used for this purpose, as it cannot be converted into plasma with this type of plasma generator. Counter electrode 116 located at the inlet end of tube 112 and high voltage electrode 118 located at the outlet end of this tube are mounted in this tube so that both are in direct contact with the gas flowing through the tube. Power source 120 applies a constant DC high voltage excitation (e.g., 8 kV) to high voltage electrode 118, which causes first plasma jet 122 to form between high voltage electrode 118 and counter electrode 116 and, in addition, a second plasma jet 124 to form between high voltage electrode 118 and grounded target 114.

As shown in FIG. 2a, DBD plasma generator 250 of this figure also includes a source of flowing gas (not shown) for directing a jet of this gas through hollow tube 252 and onto the surface of target 254 to be treated, which is also connected to ground. In addition to He other noble gases, whether at atmospheric or other pressures, as well as many other gases including N₂ and mixtures of these gases with one another can be used in this type of plasma generator. High voltage electrode 258, which is located at the outlet end of hollow tube 252, is mounted outside of this tube so that it is isolated from direct contact with the helium or other gas flowing through this tube. In addition, hollow tube 252 is made from borosilicate glass or fused silica (quartz) or other material having a high dielectric constant so that the interior of hollow tube 252 is electrically insulated from high voltage electrode 258. Power source 260 applies one or more discrete, fast rising, multiple high voltage pulses (e.g., 20 kV) to high voltage electrode 258. Alternatively, power source 260 applies a high voltage to high voltage electrode 258 in the form of a continuously operating sinusoidal waveform that continuously cycles between a large positive value (e.g., +20 kV) and a large negative value (e.g., −20 kV). As a result, plasma jet 268 forms between high voltage electrode 258 and grounded target 254.

Meanwhile, FIG. 2b illustrates another DBD plasma generator 270 which can be used to carry out this invention, which is similar to DBD plasma generator 250 of FIG. 2a, except that DBD plasma generator 270 of FIG. 2b also includes counter electrode 278 which is connected to ground. Counter electrode 278 is included in this system to make control of the electrical current easier.

As well understood in the art, a key difference between the way DC plasma generator of the type illustrated in FIG. 1 operates and the way a DBD plasma generators of the type illustrated in FIGS. 2a and 2b operate is that, in the former, a direct electrical current continuously flows through the circuit formed by this plasma generator and its associated power source while in the later no net direct electrical current flows through the circuit formed by these plasma generators and their associated power sources.

This may be more easily understood by considering FIGS. 3a, 3b and 3c, which illustrate the equivalent electrical circuits for each of these devices. As shown in FIG. 3a, the equivalent electrical circuit 310, which represents the operation of DC plasma generator 110 of FIG. 1, includes DC power source 312 as well as the component inside box 314. That is to say, the components inside box 314, including the components of FIG. 3c, represent the manner in which plasma generator 110 operates. DC power source 312 is capable of providing and maintaining a continuous high applied voltage, e.g., 8 kV, between the two electrodes of the device. As a result, a continuous direct electrical current is created and maintained in equivalent electrical circuit 310, since the plasma created by this device does allow current flow.

Meanwhile, the manner in which DBD plasma generator 250 of FIG. 2a operates is represented by equivalent electrical circuit 340 of FIG. 3b. As shown there, in addition to DBD plasma generator 250, this equivalent electrical circuit 340 also contains pulsed or alternating high voltage power source 342. This high voltage power source is different from DC power source 312 of FIG. 3a, in that power source 342 is designed to generate a high applied voltage, e.g., 2 to 40 kV, between high voltage electrode 258 and grounded target 254 by means of multiple discrete pulses or by means of a continuously alternating voltage.

As can be seen by comparing FIGS. 3a and 3b, the portions of these different equivalent electrical circuits which represent how the plasmas of these circuits operate are essentially the same, except that the components inside box 344 which define how DBD plasma generator 250 of FIG. 2a operates also include capacitor 346. Capacitor 346 is included inside box 344 to represent the capacitance developed by hollow tube 252 of this plasma generator due to its highly dielectric nature. Because of this additional capacitance, essentially no net direct current can flow through equivalent electrical circuit 340 when plasma generator 250 is operating.

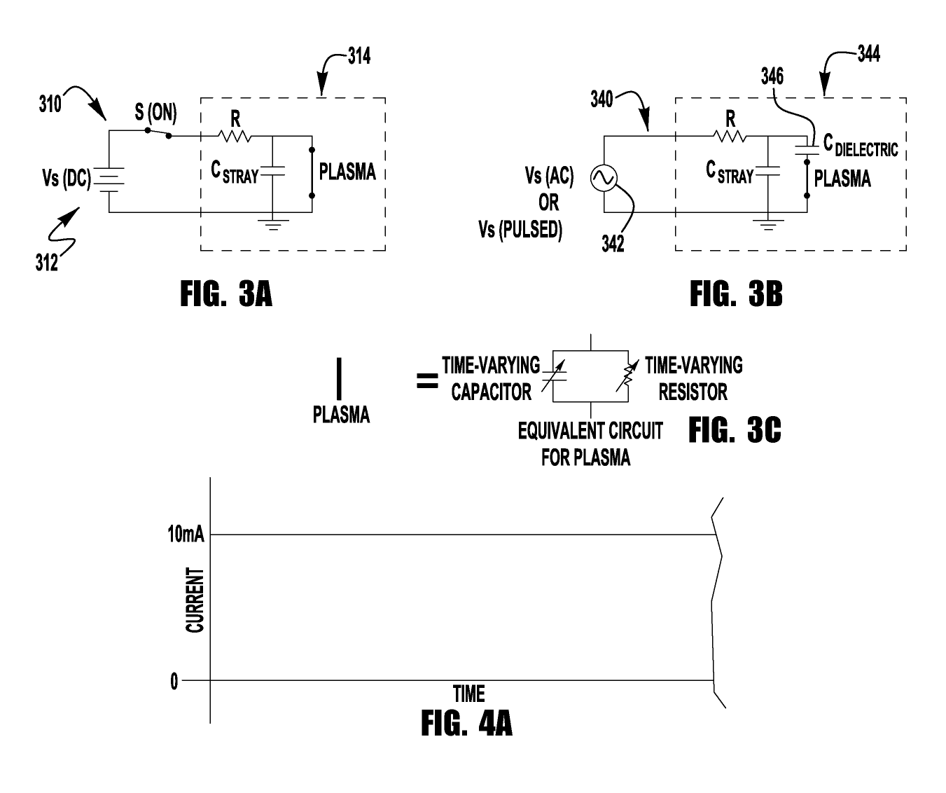

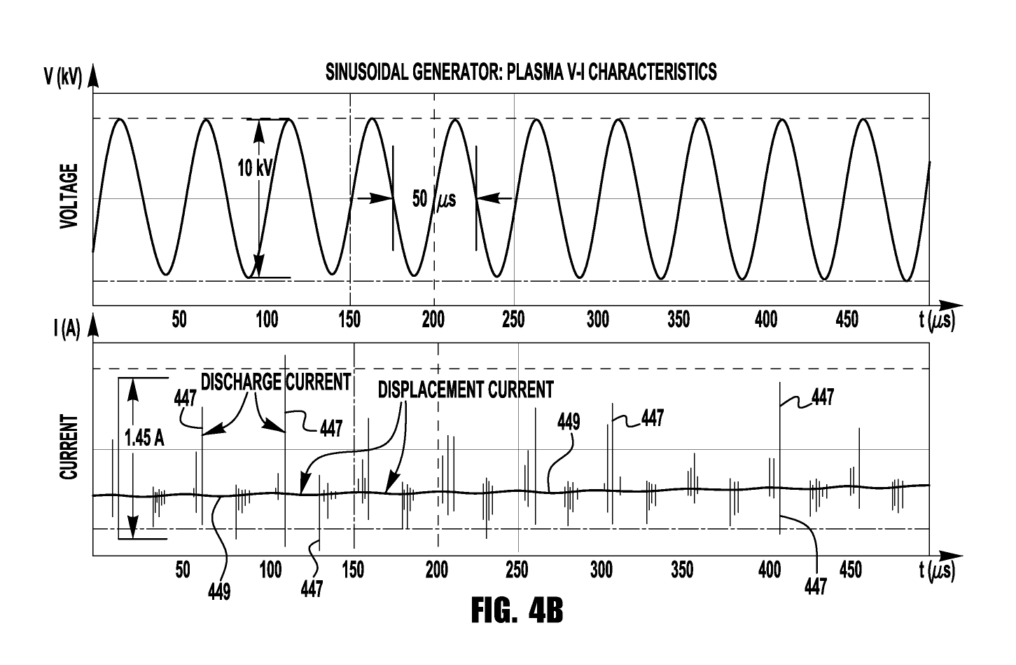

The difference in operation caused by this difference in structure can be better appreciated by considering FIGS. 4a and 4b, which are waveform diagrams illustrating the electrical currents that are created when both of these devices are operated. As shown in FIG. 4a, which illustrates the waveform of the electrical current flowing through equivalent electrical circuit 310 of DC plasma generator 110 of FIG. 1, an essentially constant electrical current of 10-100 μA flows through this circuit when this device is operating.

In contrast, as shown in FIG. 4b, which illustrates both the voltage applied and the electrical current generated in equivalent electrical circuit 340 containing DBD plasma generator 250 of FIG. 2a, the flow of electrical current through this circuit occurs only as discrete narrow spikes 447, which are labeled “Discharge current,” only twice during each period of alternating voltage, once when the voltage is rapidly increasing and again when the voltage is rapidly decreasing. This is because this flow of electrical current occurs only when a plasma is being generated, which in turn occurs only at the beginning and again at the end of a period when an alternating voltage is being used. Similarly, this flow of electrical current occurs only at the beginning and again at the end of a pulse when a pulsed voltage is being applied. At all other times, since no plasma is being generated, there is no flow of this electrical current.

As further shown in FIG. 4b, in addition to the discharge current represented by narrow current spikes 447, a so-called displacement current represented by current waveform 449 is also generated during operation of equivalent electrical circuit 340. As well understood in the electrical arts, displacement current is a measure of the rate of change of the electric field generated by a time-varying voltage. It is not formed by moving electrons, but rather by a time-varying electric field. See, Wikipedia's monograph on Displacement Current. See, also, http://www.scielo.br/scielo.php?script=sci_arttext&pid=S010397332009000300015&lng=en&nr m=iso&tlng=en.

This difference, i.e., the difference between a displacement electrical current, on the one hand, and a discharge electrical current on the other hand, is important in connection with understanding why the inventive DBD plasma generating system is so much more effective in terms of enhancing the efficacy of DNA-based vaccines than the DC plasma generator of the type shown in FIG. 1. In this regard, the discharge current represented by narrow current spikes 447 of FIG. 4b corresponds to the current flowing in equivalent electrical circuit 310 of FIG. 3a, in that both are based on moving electrons. As a result, both are capable of causing pain. In contrast, the displacement electrical current represented by current waveform 449 of FIG. 4b is not based on the flow of moving electrons and hence is not capable of causing pain. In accordance with this invention, therefore, a DBD plasma generator powered by an alternating or pulsed voltage is used to create the electrical field which creates the plasma, because by doing so that the amount of electrical current generated which is capable of causing pain is so much less. As a result, more powerful plasmas can be generated, and hence stronger electrical fields capable of inducing intracellular delivery of DNA-based vaccines, can be generated.

Incidentally, note that, as illustrated in FIG. 4b, the displacement current generated by DBD plasma generator 250 of FIG. 2a, as represented by current waveform 449, predominates over the discharge electric current that is also generated, as represented by current spikes 447. In other words, if both of these electric currents are integrated over time, the displacement electric current exceeds the conduction electric current. This same relationship, i.e., that the displacement electric current exceeds the conduction electric current over time, also occurs when a DBD plasma generator is powered by a pulsed voltage rather than an alternating voltage. Indeed, in most instances, the displacement electric current will exceed the conduction electric current by a large amount.

The practical effect of the difference between a displacement electrical current as represented by current wave form 449 of FIG. 4b and a discharge electrical current such as illustrated by the current waveform of FIG. 3a and narrow current spikes 447 of FIG. 4b relates to the fact that the equivalent electrical circuits in which both types of these devices are included also include the surface of the target to which the plasma is being applied. So, in the case of DC plasma jet generator 110 of FIG. 1, equivalent electric circuit 310 includes plasma jet 124 as well as the surface of target 114. Similarly, in the case of DBD plasma generator 250 of FIG. 2a, equivalent electric circuit 340 includes plasma jet 268 as well as the surface of target 254.

It will therefore be appreciated that, when a DC plasma jet generator of the type illustrated in FIG. 1 is used to apply a non-thermal plasma to a patient's skin, care must be taken to insure that the magnitude of the electrical current that is generated does not exceed a value which would generate pain. So, in the case of the prior art system illustrated in FIG. 1, maximum voltage was limited to 8 kV, a grounding ring connected to ground via a 1.5 GΩ resistor was needed to focus the plasma that was generated, and the power source was set to prevent current flow exceeding 100 μA although actual current flow was set to 50 μA. In addition, care also had to be taken to insure that the high voltage electrode at the outlet end of this device did not come too close to the skin being treated to prevent a spark discharge from occurring. A “spark discharge” in this context will be understood to mean a type of shorting out between the high voltage electrode and the patient's skin in which the cold plasma, in essence, transforms into a thermal plasma (arc discharge).

In contrast, when a DBD plasma generator of the type illustrated in FIG. 2a is used to apply a non-thermal plasma to a patient's skin, as occurs in this invention, many of these precautions are unnecessary. This is because the amount of current which causes pain by flowing to and across the patient's skin, which is the discharge current, is very small since it is generated only twice during each cycle or pulse in the form of very narrow spikes which are extremely short in duration.

So, as shown in FIG. 4b, the amount of current generated by the DBD plasma generator of FIG. 2 which is capable of causing pain, the discharge current represented by narrow spikes 447 of FIG. 4b, is limited in time to fractions of a microsecond if the pulse duration is in microseconds and fractions of a nanosecond if the pulse duration is in nanoseconds. In contrast, as shown in FIG. 4a, the amount of current generated by the DC plasma generator of FIG. 1, all of which is capable of causing pain, is unlimited in time, since it is constant and continuous for the entire duration of the procedure.

The practical effect of this difference is that for a given amount of plasma generation, much less electrical current of the type that can cause pain is generated when a DBD plasma generator is used in accordance with this invention than when a DC plasma generator of the type illustrated in FIG. 1 is used. As a result of this difference, in this invention, greater voltages can be used to generate the plasma which, in turn, enables stronger electrical fields to be created on the patient's skin and hence less time to complete the plasma treatment. In addition, additional steps to prevent excessive current flow such as inserting a large resistor between the patient's skin and ground and insuring that a necessary spacing is maintained between the plasma generator and the patient's skin can be avoided.

This is not to say that, in the operation of a DBD plasma generator of the type shown in FIGS. 2a and 2b, the magnitude of the discharge current spikes will always be below a level which is capable of causing pain. Indeed, the magnitude of these current spikes can reach 1 ampere and more when pulsed voltages with microsecond pulse widths are used and even 50 amperes or more when pulsed voltages with nanosecond pulse widths are used. However, even though the magnitude of these current spike might be large, the time period over which these current spikes occur is too short for the discharge current represented by these spikes to cause pain.

That is to say, in order for an electrical current generated by moving electrons to cause pain, not only does the magnitude of this current need to reach a certain level but, in addition, this current needs to flow for a period of time which is long enough to affect the tissue on which or in which the current is moving. In those situations where this period of time is so short that these moving electrons exert no effect on this tissue, no pain sensors are activated and hence no pain is generated. Accordingly, even though the magnitude of the discharge spikes generated when a DBD plasma generator is operated can reach 50 amperes and more, pain will not be generated because the time period over which the discharge current represented by these spikes is actually flowing is so short.

Based on the above, it can be seen that the structure and operation of the pulsed/alternating voltage DBD plasma generator used in this invention differ from the structure and operation of the DC plasma generator described in the above-mentioned Connolly et al. references in many important ways. As a result of these differences, many additional operating differences also occur between these systems. For example, in the case of the DC plasma generator used in the Connolly et al. publications, the voltage potential on the skin was approximately 5.6 kV and the electric field in the stratum corneum was approximately 8 kV/cm. Connolly PhD thesis, “Plasma Mediated Molecular Delivery” Pg 90, Section 4.6.3. In contrast, in the case of the DBD plasma generator of FIG. 2, when used in our invention, the voltage potential on skin was 1-15 kV, while the electric field in the stratum corneum was around 200 kV/cm. In addition, in the case of the DC plasma generator used in the Connolly et al. publications, the charge particle densities ranged from 10⁸-10¹⁰/cm³, whereas in the case of our invention, the charge particle densities ranged from 10¹²-10¹³/cm³. These substantial differences in plasma physical characteristics operating parameters further explain why the DBD plasma system of our invention is capable of generating significantly higher electric fields with significantly lower risk of pain generation than the DC plasma system of Connolly et al.

In accordance with this invention, therefore, only DBD plasma generators are used to generate the non-thermal plasmas, which are used to boost the efficacy of DNA-based vaccines in terms of eliciting a desired immune response.

In accordance with one embodiment of this invention, therefore, jet-type DBD plasma generators of the type illustrated in FIGS. 2a and 2b can be used to generate the non-thermal plasmas of this invention.

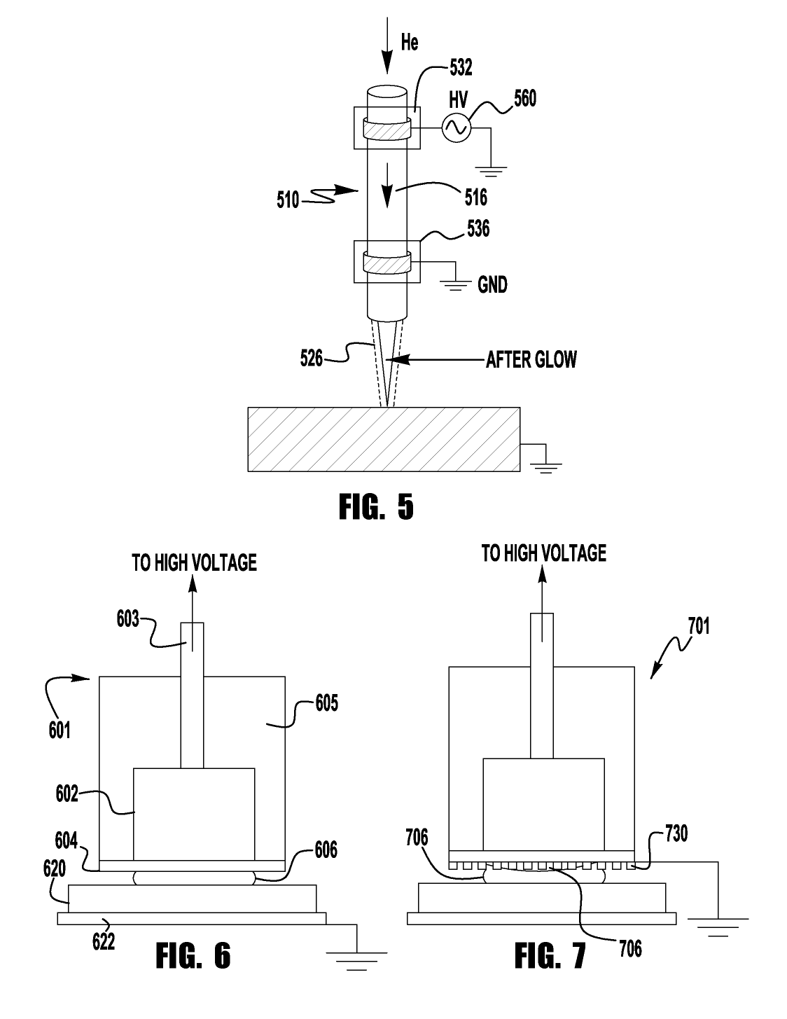

In accordance with another embodiment of this invention, a jet-type DBD plasma generator of the type illustrated in FIG. 5 can also be used to generate the non-thermal plasmas of this invention. In this regard, it is also well understood in the art of plasma generation that, not only can non-thermal plasmas be generated by two different types of plasma generators as discussed above but, in addition, two different types of non-thermal plasmas can be generated by these plasma generators. One type of non-thermal plasma is known as a “direct” or “full” non-thermal plasmas. This type of plasma contains all of the species that are created when a cold plasma is generated, i.e., charged ions, electrons, reactive oxygen and nitrogen species and excited neutral atoms. The DC plasma generators of FIGS. 1, 2a and 2b create this type of non-thermal plasma.

In the other type of non-thermal plasma, essentially all of the electrons and charged ions have been removed, leaving behind only the neutral reactive oxygen and nitrogen species and neutral atoms and molecules to remain in the plasma. Such non-thermal plasmas are known as “indirect” or “afterglow” plasmas. FIG. 5 illustrates still another type of DBD plasma generator that can be used to generate a cold plasma for use in this invention, the non-thermal plasma created by this DBD plasma generator being an indirect non-thermal plasma.

As shown in FIG. 5, indirect non-thermal plasma generator 510 has essentially the same structure as direct non-thermal plasma generator 270 of FIG. 2b, except that in plasma generator 510, the electrical connections to the high voltage power source and ground are reversed. So, in non-thermal plasma generator 510, high voltage electrode 532, which is connected to high voltage power source 560, is mounted on the outside of tube 512 near its inlet end rather than its outlet end. In addition, counter electrode 536, which connected to ground, is mounted on the outside of tube 512 near its outlet end rather than its inlet end. As a result, plasma 516 is created inside of tube 512 between high voltage electrode 532 and counter electrode 536. Counter electrode 536 does not remove charged particles that are created in this plasma, as occurs during the operation of other types indirect non-thermal plasma generators. Rather, counter electrode 536 in combination with the high voltage pulsed or continuous AC excitation generated by high voltage power source 560 causes these charged particles to be trapped inside tube 512, which acts like a capacitor due to its highly dielectric nature. The result is that only neutral particles remain in plasma 526 which are pushed out of the tube due to the flow of gas being used to generate the plasma. Nonetheless, it is believed that plasma 526 can still be used for carrying out this invention.

Incidentally, because the area over which plasma is delivered to the surface of a target being treated by jet type plasma generators is so small, typically about 10 mm² or so, if a jet-type plasma generator is used for carrying out this invention, an assembly mounting this plasma generator can optionally be provided to enable rastering of this plasma generator for covering a larger area of the surface being treated.

FIGS. 6 and 7 illustrate still additional types of DBD plasma generators that can be used for carrying out this invention. Both of these DBD plasma generators are so-called “large area” plasma generators, meaning that that they are capable of generating a plasma essentially uniformly over an area of at least 2 cm². Many large area plasma generators are capable of generating plasmas essentially uniformly over an area of at least 7.5 cm², at least 10 cm², at least 12.5 cm², at least 15 cm², or at least 20 cm². In contrast, the plasma generated by a typical plasma jet generator has an area of 10 mm² or less, as indicated above.

FIG. 6 schematically illustrates the type of large area DBD plasma generator that is designed to generate a full or direct non-thermal plasma. As shown there, plasma generator 601 includes a high voltage source (not shown), conductor 603, housing 605, high voltage electrode 602 and dielectric barrier 604. In the particular embodiment shown, plasma generator 601 is mounted a suitable distance, e.g., 2 mm, above the surface of target 620 to be treated, e.g., skin. Target 620 serves as the counter electrode, as it may be grounded as shown in this figure, or it may be a floating ground, i.e., ungrounded. During operation, the high voltage source is turned on and plasma 606, which forms between the dielectric barrier 604 and the surface of target 620 and which contains all three of electrons, ions and neutrals, treats the surface of target 620. As a result, a strong electric field is instantly generated on this surface and in the substrate due to the deposition of these charged particles on this surface.

Meanwhile, FIG. 7 schematically illustrates the type of large area DBD plasma generator that is designed to generate an indirect non-thermal plasma. As shown there, plasma generator 701 has essentially the same structure as large area DBD plasma generator 601, except that plasma generator 701 further includes filter 730 in the form of a conductive mesh which serves as the counter or grounding electrode of the device. Plasma generator 701 operates in much the same way as plasma generator 601, except the plasma is generated between the dielectric barrier 704 and the grounded filter 730 which means charged ions and electrons are prevented from passing through grounded filter 730. It will therefore be appreciated that the modified plasma 706 which is obtained is also an “afterglow” or “indirect” plasma, like indirect plasma 526 generated by plasma generator 510 of the system of FIG. 5. Because plasma 706 is an indirect plasma, no charges are deposited on the target and only neutral species and energetic reactive oxygen and nitrogen species come in contact with the substrate.

### Power Source

As further discussed below, an important feature of this invention is that the plasma which is directed at the target being treated is generated by the application of a pulsed or alternating high voltage rather than a constant voltage. As of this writing, there are basically three different types of commonly-available power sources which can generate the pulsed or alternating electric voltages of interest in this invention, (1) those capable of generating high voltage pulses having microsecond pulse widths, (2) those capable of generating high voltage pulses having nanosecond pulse widths and (3) those capable of generating alternating high voltages in a simple sinusoidal wave form. Because the terms commonly used to describe the details of electric power can have different meanings depending on the type of power source used, we present this section to make clear what the terms we use in this disclosure mean as they relate to each of these different types of power sources.

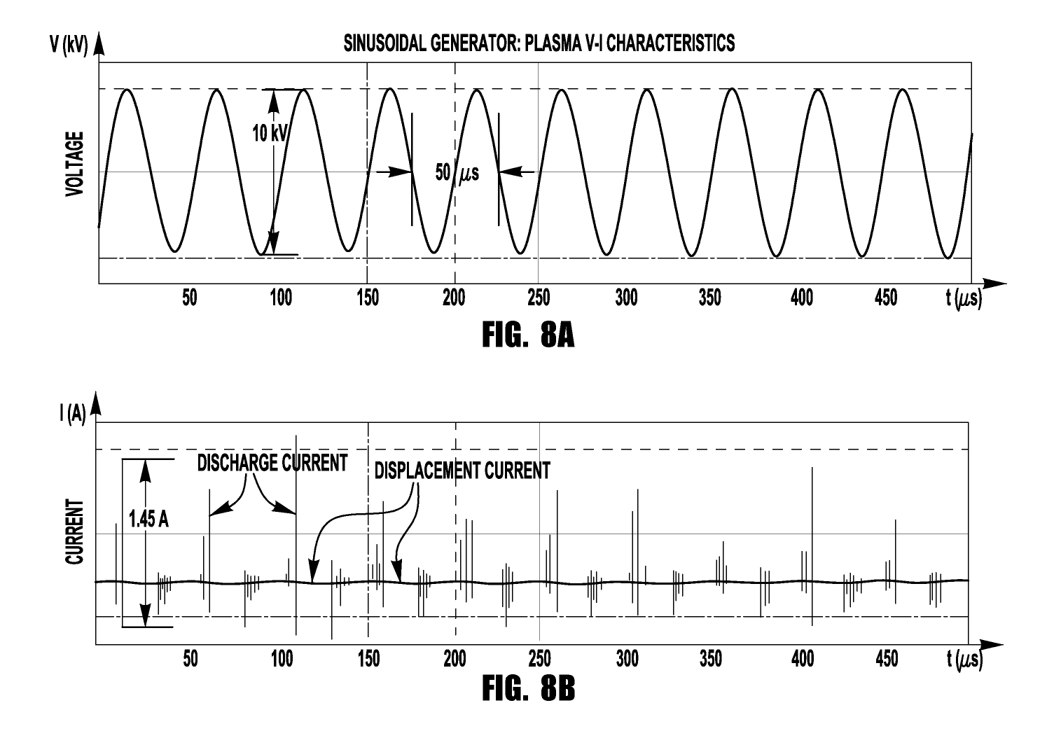

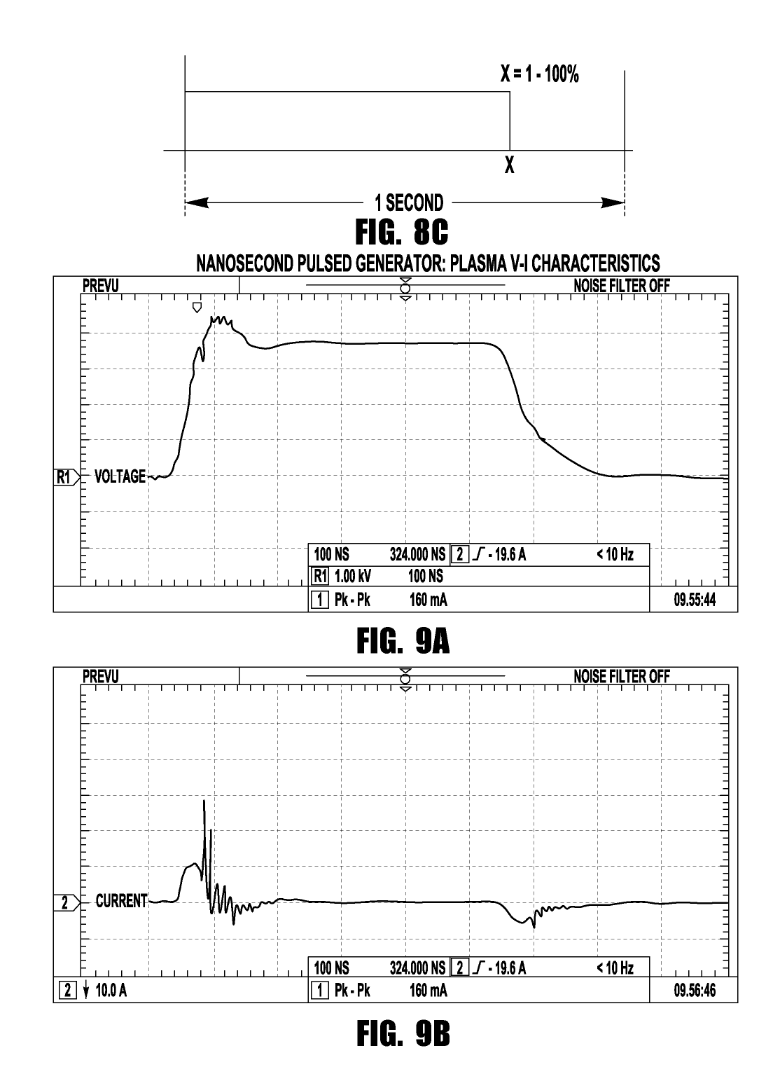

FIG. 8a illustrates the voltage waveform generated by a typical power source capable of generating voltage pulses having microsecond pulse widths. Such machines are typically capable of generating pulses at voltages from 1 to 30 kV or more, with pulse widths of 1-100 μs, at frequencies of 1 Hz to 20 kHz, more typically 50 Hz to 10 kHz, and even more typically 500 Hz to 2.5 kHz at duty cycles of 1-100%. Typical rise times for pulses generated by such devices range between 0.01 V/ns and 20 V/ns. FIG. 8a illustrates a typical voltage waveform generated by such a device when set to provide pulses having a voltage drop of 23 kV peak-to-peak (between −11.5 kV and +11.5 kV) and a pulse width of 10 μs at a frequency of 1 kHz. In accordance with conventional practice, this pulse width will be understood to refer to the width of the pulse when at half of its maximum value, i.e., the so-called full width half maximum (“FWHM”) pulse width, which in this case is 5 μs.

Meanwhile, FIG. 8b illustrates the associated current waveform that is produced when the power source of FIG. 8a is powering a DBD plasma generator of the type used in this invention. As shown in this figure, the associated current waveform that is generated, like that of FIG. 4b, also is defined by periodic extremely narrow current spikes representing discharge current as well as a continuous current waveform representing displacement current.

FIG. 8c illustrates what we mean by “duty cycle” in connection with the power source of FIG. 8a. As shown there, the duty cycle of such a power source refers to the portion of time during the operation of the machine when a signal is actually being generated. So, if the duty cycle X is 75%, for example, this means that, for each second of operation, the signal represented by FIG. 8a is being generated by the machine for 75% of that second (i.e., ¾ second) while no signal is being generated by the machine for the remaining 25% of that second. This means that, as used here, “duty cycle” is independent of period. Also note that, in keeping with conventional practice, where nothing is said about duty cycle, it will be understood that the duty cycle is 100%.

FIG. 9a illustrates the voltage waveform generated by a typical power source capable of generating pulses having nanosecond pulse widths. Such machines are typically capable of generating pulses at voltages from 1 to 40 kV or more, with pulse widths of 1-999 ns, more typically 1-500 ns. Such machines can be set up for manual operation in which each pulse is activated manually, such as by pushing a button. In addition, such machines can be set up for automatic operation, in which case they can operate at frequencies typically ranging from 1 Hz to 20 kHz. Typical rise times for pulses generated by such devices range between 0.5 and 10 kV/ns. Because the pulse widths generated by these machines are so short and further because these pulses can be initiated manually, these machines are not normally set up to allow operation at duty cycles of less than 100%.

FIG. 9a illustrates the voltage waveform generated by such a device when set to provide pulses having a maximum voltage of 17 kV and a pulse width of 500 ns. As shown there, each pulse takes the form of a trapezoid whose amplitude rapidly rises to a 20 kV peak which then rapidly settles to the target voltage of 17 kV where it remains essentially constant until the end of the pulse, at which time the amplitude rapidly decreases back down to zero. PW in FIG. 9a also refers to the pulse width of the pulse that is generated, and as in the case of the microsecond power source of FIG. 8a, PW in the case of the nanosecond power source of FIG. 9a also refers to the width of this pulse when at half of its maximum value, i.e., the so-called full width half maximum (“FWHM”) pulse width. As shown in FIG. 9b, the associated current waveform generated when the nanosecond power source of FIG. 9a is used to drive a DBD plasma generator in accordance with this invention, like that of FIGS. 4b and 8b, also is defined by periodic extremely narrow current spikes representing discharge current as well as a continuous current waveform representing displacement current.

FIG. 10 is a schematic diagram illustrating a typical power source capable of generating simple alternating voltage waveforms. Insofar as relevant to this invention, such machines are capable of generating alternating electrical currents which generate voltage waveforms in the form of continuously-operating sinusoidal waves which continuously cycle between a large positive value and a large negative value. Voltages of from 1 to 40 kV at frequencies of 1 to 30 kHz can be used. FIG. 10 illustrates the voltage waveform generated by such a device when set to provide alternating electrical current which generates maximum and minimum voltages of +5 kV and −5 kV, respectively, at a frequency of 20 kHz, with the period of this waveform being 1/f=50 μs.

### Intracellular Delivery of DNA-Based Vaccines

As indicate above, the primary focus of this invention is on facilitating the delivery of DNA-based vaccines from at or near the cells to be treated through the cell walls of these cells into their interiors. For convenience, we refer to this as an “intracellular” delivery of these vaccines. This terminology is intended to distinguish processes in which a non-thermal plasma is used to facilitate the transdermal delivery of such vaccines, i.e., the delivery of such vaccines through the skin from outside the patient's body to inside the patient's body, which we refer to as an “intercellular” delivery of these vaccines.

In accordance with this invention, we have found that the application of a non-thermal plasma which has been generated by a DBD plasma generator to the site where a DNA-based vaccine has previously been transdermally delivered, either by injection or topical application followed by migration through the patient's skin, will greatly facilitate the uptake of the drug into the interiors of the target cells being treated and hence the efficacy of the vaccine as a whole in terms of providing a desired immune response, even though this vaccine does not “pass through” the non-thermal plasma as required by the above-noted Jacofsky et al. publication.

### DNA-Based Vaccines

DNA vaccination is a technique for protecting an animal against disease by injecting it with genetically engineered DNA so that immune cells in the animal's body directly produce an antigen, resulting in a protective immunological response. As of this writing, several DNA-based vaccines have already been released for veterinary use, while one has been released in Japan for human use. In addition, significant research is ongoing in connection with using these vaccines for treating infectious diseases that are caused by viral, bacterial and parasitic infections, as well as various cancers.

DNA-based vaccines are third generation vaccines. They contain plasmid DNA encoding specific proteins (antigens) from a pathogen or tumor. When the vaccine is injected into the body, certain types of host cells found in the body engulf and read the DNA and use it to synthesize the pathogen's proteins. Because these proteins are foreign to the cell, they become displayed on its surfaces. This alerts the immune system, which then triggers an appropriate immune response.

DNA-based vaccines can be applied to a subject using a variety of different conventional treatment protocols, all of which involve the injection of the vaccine into the patient's body. For example, they can be applied by a means of a single injection or multiple injections normally spaced apart by several days or even weeks. In addition, when multiple applications are involved, variations in dosage levels are also possible. Thus, in some situations, the same amount of the same parenteral composition will be injected. In other situations, lesser amounts of the DNA-based vaccine can be used in one or more of subsequent injections (so-called “booster shots”), either by using less parenteral composition, a less-concentrated parenteral composition, or both. In still other embodiments, one or more injections of the DNA-based vaccine can be followed by one or more injections of the target protein, i.e., the antigen produced by host cells inoculated with the DNA-based vaccine. In still other embodiments, the DNA-based vaccines can be applied topically rather than by injection.

Regardless of the particular vaccination protocol being used, it is well-understood in the art that DNA vaccines by themselves are not very effective, as they are not efficiently taken up by the immune cells. To address this problem, electroporation is currently being studied to open up the target cells for better uptake of the vaccines. In this regard, electroporation is similar to the plasma treatments described here and in the earlier prior art references mentioned above except that, in electroporation, the electrodes which generate an electric field across the surface of a patient's are in direct contact with this skin. As a result, various drawbacks occur including pain, muscle contractions, skin irritation etc. In accordance with this invention, plasma-assist technology using a DBD plasma generator powered by a pulsed or alternating high voltage source is used for enhancing the efficacy of DNA-based vaccine.

In carrying out this invention, any DNA-based vaccine which has been used in the past or which may be developed in the future can be used. Similarly, this invention can also be used for enhancing the efficacy of any other vaccine based on analogous genetic materials such as RNA and XNA which have been used in the past or which may be developed in the future. In addition, this plasma-assist technology can also be used in connection with any vaccination protocol which has been used in the past or which may be developed in the future for delivering vaccines based on DNA and/or other genetic materials.

### Operating Details

In accordance with this invention, any combination of DBD plasma generator and power source which is capable of generating a cold plasma can be used to enhance the efficacy of a DNA-based vaccine which has previously been delivered into the patient's body.

However, for best results, we have found it preferable to select the particular plasma treatment to be used in particular embodiments of this invention based on the specific type of power source that will be used.

For example, in those instances in which the power source to be used is designed or set up to supply high voltage pulses having nanosecond pulse widths such as illustrated in FIG. 9 it is desirable to operate at voltages that range from 2 to 40 kV. Within such a range, minimum voltages of at least 3 kV, at least 5 kV, at least 10 kV, at least 15 kV and at least 18 kV are contemplated, as are maximums of 35 kV, 30 kV, 25 kV and 22 kV. Voltage ranges of 3 to 30 kV, more typically 10-28 kV, 15-25 kV and even 18-22 kV are also contemplated.

The pulse widths of the pulses used in this embodiment of the invention can range anywhere between 30 and 999 ns. Thus, pulse widths of at least 50 ns, at least 100 ns, at least 150 ns, at least 200 ns, at least 250 ns, at least 300 ns, at least 350 ns and at least 400 ns are contemplated. Similarly, maximum pulse widths of 950 ns, 900 ns, 850 ns, 800 ns, 750 ns, 700 ns, 650 ns, 600 ns and 550 ns are contemplated. In specific embodiments, pulse widths of 30-500 ns, 50-400 ns, 100-300 ns, 150-250 ns and 175-225 ns are contemplated as are pulse widths of 75-600 ns, 100-500 ns and 150-300 ns.

Two basic modes of operation are contemplated when such a power source is used, one using manual pulse activation, the other using automatic pulse activation. In this context, “manual” pulse activation will be understood to mean an operation in which each pulse is activated manually, such as by the push of a button. The operating regimes used in Groups 7 and 8 in the following Example 1 and Groups 3 and 4 in the following Example 2 are examples of manual pulse activation in the context of this disclosure.

When manual pulse activation is used, the total number of pulses involved in a particular plasma treatment or regimen will generally not exceed 100. More commonly, the total number of pulses involved in a particular plasma treatment will not exceed 75, 45, 40, 35, 30 or even 25 pulses. In this regard, note that in the following examples, very effective results are achieved with as few as 20 pulses. In addition, we have determined that effective results can be achieved with even a single pulse, although using at least 5, at least 10 or at least 15 pulses would be more typical. Thus, it is contemplated the total pulses that will be used in a single plasma treatment or application, whether done in one part or two parts, can range from 5 to 100, 10 to 60 and even 20 to 50.

In automatic pulse activation, the power source is designed and set to generate pulses automatically. Frequencies can be as little as 2 Hz and as much as 20 kHz. Typical frequency ranges include 100 Hz to 20 kHz, 500 Hz to 10 kHz, 2 kHz to 5 kHz. If automatic pulse activation is used, the total time over which the plasma treatment occurs will normally be no more than 3 minutes, more normally no more than 2.5 minutes, 2 minutes, 1.5 minutes or even 1 minute. Longer treatment times can be used, if desired. However, as can be seen from the following working examples in this disclosure that excellent results are achieved when automatic pulsing lasted either 60 or 30 seconds.

In another mode of automatic pulse activation, a discrete number of pulses is automatically applied using an external trigger. From 1-100,000 pulses could be externally applied in this manner as a train of pulses.

Weather manual or automatic pulsing is used, it will be understood that the rise time of the individual pulses in this embodiment of the invention in which nanosecond pulses are used will be very short, because the pulse widths of these nanosecond pulses is so short. Thus, rise times on the order of 0.5-10 kV/ns, 1-8 kV/ns or even 2-6 kV/ns are typical.

In some situations in which nanosecond pulses are used, it may be desirable to use combination plasma treatments. In such a combination plasma treatment, the plasma treatment or regimen is divided into a first part and a second part which differ from one another in that a lower voltage and a longer pulse width are used in the second part relative to the first part, or conversely. In such pulsing regimens, the difference between the voltages used in the first and second parts will normally be at least 5 kV, but can also be as much as 6 kV, 7 kV or even 8 kV, while the pulse widths will differ by a factor of at least 2 (e.g., 200 vs. 100 ns) but may differ by as much as a factor of 3, 4 or 5.

An example of one specific combination plasma treatments being contemplated includes a regimen in which the applied voltage in the second part is at least 5 kV less than the applied voltage in the first part and the pulse width of pulses in the second part is at least twice as long as the pulse width in the first part. In this regimen, the operating conditions in the first part are contemplated to be 1-50 pulses, applied voltages of 20-30 kV and pulse widths of 20-100 ns, while the operating conditions in the second part are contemplated to be 1-50 pulses, applied voltages of 3-19 kV and pulse widths of 120-999 ns.

A second specific combination plasma treatment being contemplated involves conditions which are essentially the reverse of those used in the first combination. In this second combination, the applied voltage in the second part is at least 5 kV more than the applied voltage in the first part and the pulse width of pulses in the second part is half or less as long as the pulse width in the first part. In this regimen, the operating conditions in the first part are contemplated to be 1-50 pulses, applied voltages of 3-19 kV and pulse widths of 120-999 ns, while the operating conditions in the second part are contemplated to be 1-50 pulses, applied voltages of 20-30 kV and pulse widths of 20-100 ns.

In the particular combination treatment described here, as well as all other combination treatments described in this disclosure, the time elapsed between the two parts of the combination treatment range anywhere from a minimum of no time elapse (i.e., second part is carried out immediately after the first part) to 5 hours or more. More commonly, the time elapse will range between 30 seconds to 5 minutes, 1 to 4 minutes or 2 to 3 minutes.

Turning now to those instances in which the power source to be used is designed or set up to supply high voltage pulses having microsecond pulse widths such as illustrated in FIG. 8a, —it is also desirable to operate at voltages that range from 2 to 40 kV. Within this range, minimum applied voltages of at least 3 kV, at least 5 kV, at least 10 kV, at least 15 kV and at least 18 kV are contemplated, as are maximums of 35 kV, 30 kV, 25 kV and 22 kV. Applied voltage ranges of 3 to 30 kV, more typically 10-28 kV, 15-25 kV and even 18-22 kV are also contemplated.

The pulse widths of the pulses used in this embodiment of the invention can range anywhere between 1 to 50 μs, although pulse widths of 1 to 30 μs, 1 to 20 μs, 1 to 15 μs, 1 to 10 μs, 1 to 7.5 μs or even 1 to 5 μs will be more common. Because these pulse widths are longer in time, pulse rise times will generally also be longer. Thus, typical rise times will likely be 1 to 20 V/ns, 2 to 12 or even 4 to 8 V/ns.

Pulses of this type will normally be supplied at frequencies ranging from 50 Hz to 5 kHz, more typically 400 Hz to 3.5 kHz, 500 Hz to 2.5 kHz at duty cycles of 1-100%. Duty cycles of at least 50%, at least 60%, at least 70%, at least 80%, at least 90% and at least 95% are more common. Duty cycles of 100% will often be used. In this regard, as indicated above, when nothing is said about the duty cycle of a particular mode of operation using automatic pulsing, the duty cycle will be understood to be 100%, in accordance with conventional practice.

With respect to treatment times, when pulses with microsecond pulse widths are used, the total treatment time for a particular plasma application will generally not exceed 2 minutes, although longer treatment times can be used if desired. More commonly, total treatment times will not exceed 135 seconds, 90 seconds, 75 seconds, 60 seconds, 45 seconds or even 30 seconds.

As in the case of using voltage pulses with nanosecond pulse width, it is also contemplated that when voltage pulses with microsecond pulse widths are used in accordance with this embodiment, combination plasma treatments can also be used. Like the combination plasma treatments discussed above, the combination plasma treatments used in this embodiment can also be composed of a first part and a second part. In this instance, however, the different parts differ from one another in that a lower applied voltage and a lower frequency are used in the second part relative to the first part, or conversely. In such pulsing regimens, the difference between the applied voltages used in the first and second parts will normally be at least 5 kV, but can also be as much as 6 kV, 7 kV or even 8 kV, while the frequencies will differ by a factor of at least 30 (e.g., 100 Hz vs. 3,000 Hz) but may differ by as much as a factor of 35, 40 or even 45.

An example of one specific combination plasma treatments being contemplated for use with microsecond pulse widths includes a regimen in which the applied voltage in the second part is at least 5 kV less than the applied voltage in the first part and the pulse width of pulses in the second part is at least twice as long as the pulse width in the first part. In this regimen, the operating conditions in the first part are contemplated to be applied voltages of 20-30 kV, pulse widths of 1 to 25 μs, and frequencies of 500-20000 Hz, while the operating conditions in the second part are contemplated to be applied voltages of 3-19 kV, pulse widths of 2 to 50 μs, and frequencies of 1-500 Hz.

A second specific combination plasma treatment being contemplated involves conditions which are essentially the reverse of those used in the first combination. In this second combination, the voltage applied voltage in the second part is at least 5 kV more than the applied voltage in the first part and the pulse width of pulses in the second part is half or less as long as the pulse width in the first part. In this regimen, the operating conditions in the first part are contemplated to be applied voltages of 3-19 kV and pulse widths of 2 to 50 μs, and frequencies of 1-500 Hz, while the operating conditions in the second part are contemplated to be applied voltages of 20-30 kV and pulse widths of 1 to 25 μs, and frequencies of 500-20000 Hz.

A third specific combination plasma treatment being contemplated involves a first treatment using a microsecond pulsed generator and the second treatment using a nanosecond pulsed plasma generator. For example 2500 Hz, 5 μs, 17 kV for 30 s followed by 20 kV, 500 ns, 50 pulses or 2500 Hz, 5 μs, 17 kV for 30 s followed by 20 kV, 200 Hz, 200 ns for 1 min or vice versa (nanosecond first and microsecond next)

Turning now to power sources designed or set up to supply high voltage power in the form of simple alternating voltage waveforms, it is also desirable to operate at voltage amplitudes that range from 2 to 40 kV. In other words, the voltage can cycle between +2 kV to −2 kV, between +40 kV to −40 kV, and anywhere in between. Within this range, minimum voltages of at least +3/−3 kV, at least +5/−5 kV, at least +10/−10 kV, at least +15/−15 kV and at least +18/−18 kV are contemplated, as are maximums of +35/−35 kV, +30/−30 kV, +25/−25 kV and +22/−25 kV. Voltage ranges of +3/−3 to +30/−30 kV, more typically +10/−10-+28/−28 kV, +15/−15-+25/−25 kV and even +18/−18-+22/−22 kV are also contemplated.

In addition, it is also desirable to operate such power sources at frequencies of 1 to 30 kHz, 3 to 20 kHz, 5 to 15 kHz and 7 to 12 kHz.

Finally, regardless of which of the above types of operating regimen is used, it is desirable for avoiding patient discomfort to operate so that the energy deposited on intact skin is less than about 100 J/cm², more typically less than about 50 J/cm², less than about 20 J/cm², or even less than about 10 J/cm².

### Vaccination Protocol

The simplest way of carrying out the inventive process is to deliver a DNA-based vaccine to inside a patient's body a single time and then apply the plasma-assist technology of this invention a single time to the application site on the patient's skin where the DNA-based vaccine was delivered.

However, as indicated above, it is well known that many different vaccination protocols can be used to deliver vaccines to inside a patient's body including using multiple injections of the same vaccine as well as using one or more injections of a particular vaccine in combination with one or more injections of the target protein, i.e., the protein that that particular vaccine has been designed to produce. In this regard, for convenience, in this disclosure we use the term “prime” to refer to the delivery inside a patient's body of a particular DNA-based vaccine and the term “boost” to refer to the delivery inside the patient's body of the protein that the particular vaccine has been designed to produce, which in the case of this invention is the protein encoded by that particular DNA-based vaccine.

In accordance with this feature of the invention, the inventive plasma-assist technology is used in combination with a vaccination protocol involving multiple deliveries, i.e., a vaccination protocol in which a particular DNA-based vaccine is delivered at least once and one or more additional deliveries are made either of the same DNA-based vaccine, the target protein encoded by the DNA-based vaccine, or both.

In this embodiment of the invention, each time a DNA-based vaccine or protein is delivered to a patient's body, this delivery can be followed by the application of the plasma assist technology of this invention to the site on the patient's skin where this delivery was made. Alternatively, the plasma assist technology of this invention can be applied to less than all of these deliveries. For example, the plasma assist technology of this invention can be applied only to deliveries of DNA-based vaccines, but not to deliveries of the corresponding protein. Similarly, the plasma assist technology of this invention can be applied to less than all of the deliveries of DNA-based vaccines, for example only to the first delivery, or only to the first and second delivery. Alternatively, a single DNA injection could be followed by multiple plasma treatments. Regardless of how many times the plasma assist technology of this invention is applied, it is believed that the efficacy of the DNA-based vaccine in terms of eliciting a desired immune response will be significantly improved.

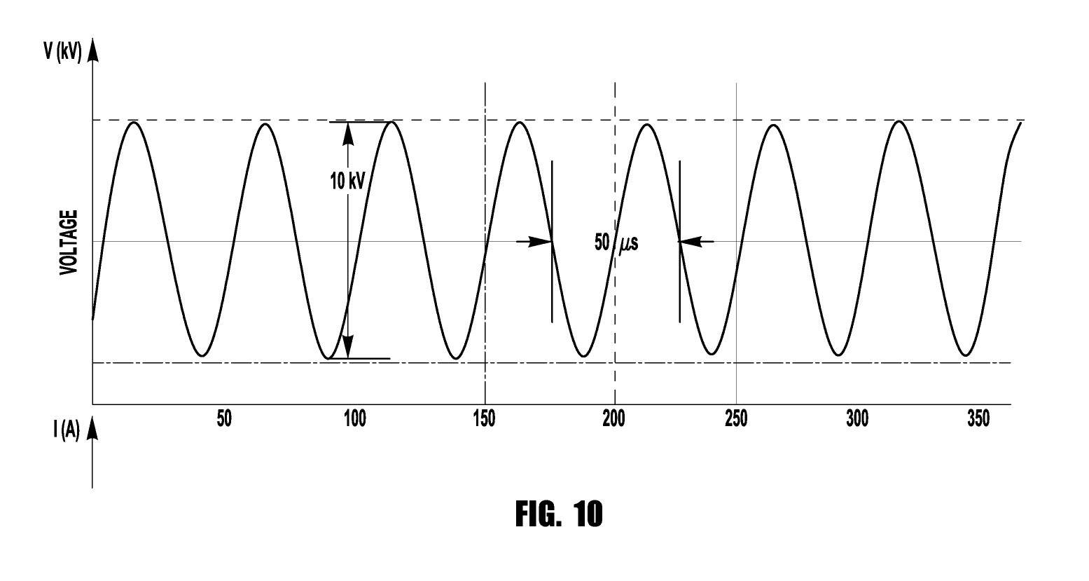

FIGS. 11a and 11b are block diagrams which illustrate two examples of this embodiment of this invention. In particular, FIG. 11a illustrates the vaccination protocol carried out in the following Example 1 of this disclosure in which two injections were made, both involving the DNA-based vaccine of interest, while FIG. 11b illustrates the vaccination protocol carried out in the following Example 2 of this disclosure in which two injections were made, the first involving the DNA-based vaccine of interest and the second involving the corresponding protein encoded by that DNA-based vaccine. Note that, in the vaccination protocol of FIG. 11a, the plasma assist technology of this invention was applied to both deliveries of the DNA-based vaccine of interest. In contrast, in the vaccination protocol of FIG. 11b, the plasma assist technology of this invention was not applied to the “boost” site of this protocol, i.e., the site where the protein encoded by the DNA-based vaccine was injected. This is because the protein of this injection was not intended to be delivered to inside the target cells of interest.

Although the particular vaccination protocols used in these diagrams and examples involved only two deliveries spaced 14 days apart followed by analysis of the ultimate results obtained on day 28, it will be appreciated that many different vaccination protocols can be used involving different numbers of vaccine and/or protein deliveries as well as different delay periods between successive deliveries. For example, 3, 4 or even 5 different deliveries can be made, each involving the DNA-based vaccine of interest only or, alternatively, some (including the first) involving delivery of the DNA-based vaccine and others involving delivery of the protein encoded by the vaccine. In addition, in those cases in which both the DNA-based vaccine and its corresponding protein are delivered, they can be delivered alternately, i.e., each vaccine injection is followed by a protein injection. Alternatively, all the vaccine can be delivered before any protein is delivered.

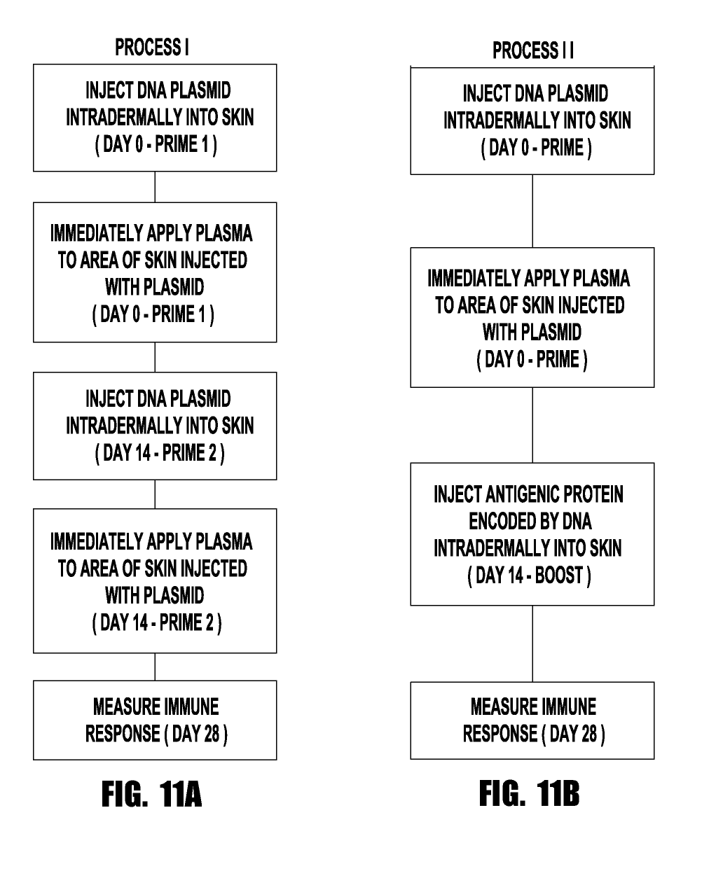

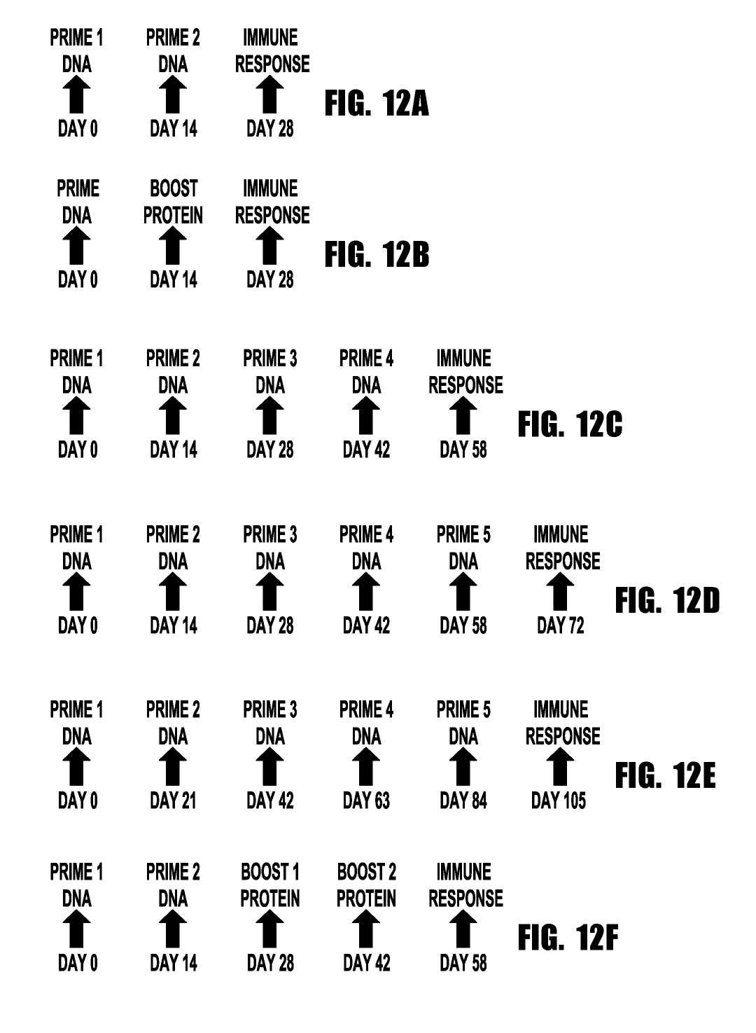

In this regard, FIGS. 12a-12f are schematic diagrams which illustrate a number of different vaccination protocols which are possible in accordance with this invention. FIGS. 12a and 12b illustrate the vaccination protocols used in the following Examples 1 and 2, respectively, which are described above in connection with FIGS. 11a and 11b. Meanwhile, FIGS. 12c and 12d illustrate similar vaccination protocols involving 4 and 5 injections of the DNA-based vaccine of interest, while FIG. 12e illustrates a similar vaccination protocol involving 5 injections of the DNA-based vaccine of interest which are spaced apart by 21 days instead of 14 days. Finally, FIG. 12f illustrates a vaccination protocol in which two injections of the DNA-based vaccine of interest are followed by two injections of the protein encoded by that DNA-based vaccine.

### Plasma-Assisted Intercellular Delivery of DNA-Based Vaccines

As indicated above, the primary focus of this disclosure is on the plasma-assisted intracellular delivery of DNA-based vaccines, i.e., the delivery of such vaccines from a site which is inside a patient's body at or near but outside of the cells to be treated into the interior of these cells. As further indicated above, the delivery of these vaccines from an application site outside the patient's body to inside the patient's body will normally be done by injection. However, in accordance with this optional feature of this invention, this delivery of these vaccines from outside to inside the patient's body can also be done by topical application, i.e., by applying these vaccines to the surface of the subject's skin and allowing them to migrate to inside the patient's body by natural phenomena, provided that before this vaccine is topically applied a non-thermal plasma is applied to the application site where this DNA-based vaccine will be topically applied.

In this regard, in commonly-assigned application U.S. 2015/0094647 (35416/04025), the disclosure of which is incorporated herein by reference in its entirety, a non-invasive method is described for facilitating the transdermal delivery of topically-applied drugs and other molecules from outside to inside a patient's body by exposing the patient's skin or tissue to a non-thermal plasma before the drug is applied. As described there, the effect of this non-thermal plasma pre-treatment, which is referred to there as “plasmaporation,” is to open pores is the subject's skin, thereby making it far more permeable to the passage of drugs and other molecules that are normally unable to diffuse through on their own. The overall result is that the rate at which particular drugs and other molecules can be delivered through the skin is greatly enhanced. In addition, the maximum size of molecules which can be delivered transdermally is also greatly increased. As previously indicated, we refer to this type of drug delivery as an “intercellular” delivery.

Meanwhile, in parent application US 2015/0151135 (Atty. Docket No. 35416/04028), this technique is specifically described as being useful for the intercellular delivery of DNA-based vaccines.

In accordance with this optional feature of this invention, this intercellular transdermal delivery technique for delivering a drug from outside to inside a patient's body is used to deliver the DNA-based vaccines of interest in this invention to inside the patient's body before the plasma-assisted technology of this invention is carried out.

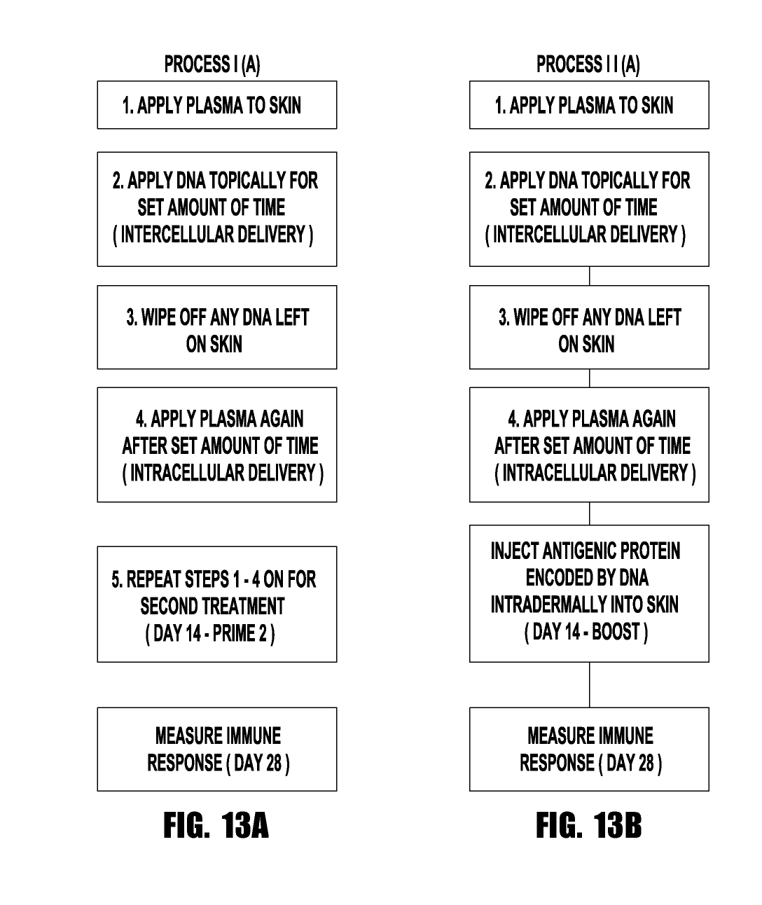

This optional embodiment of this invention is illustrated in FIGS. 13a and 13b. As shown in FIG. 13a, the inventive process when using this option feature starts in step 1 with the application of a cold plasma to the application site on the patient's skin where the DNA-based vaccine is to be topically applied. The DNA-based vaccine is then topically applied in step 2, after which any excess can be wiped off, if desired, in optional step 3. Then, in step 4, the plasma-assist technology of this invention is used by applying a cold plasma to the application site where the DNA-based vaccine was applied, thereby enhancing the intracellular delivery of the vaccine to inside the target cells of interest.

In this regard, note that it is desirable to allow a certain period of time to elapse before cold plasma treatment of step 4 begins. The primary purpose of this delay is allow sufficient time for the topically applied DNA-based vaccine to migrate to inside the patient's body Moreover, as discussed above in connection with the above-noted Jacofsky et al. disclosure, application of a cold plasma directly to a DNA-based vaccine runs the risk of destroying and/or inactivating the vaccine due to the ability of cold plasmas to readily oxidize many different materials. Therefore, a secondary purpose of this delay is enable the topically applied DNA-based vaccine to migrate away from the surface of the skin so that it is not directly contacted with the cold plasma applied in step 4. Delay times can be as short as 30 seconds, although such delay times will normally be at least about 1 minute, at least about 2 minutes, at least about 5 minutes, at least about 10 minutes and even longer.

Once the DNA-based vaccine migrates into the patient's body, step 4 is carried out to induce intracellular delivery of the DNA-based vaccine to inside the target cells of interest. Then, as further illustrated in FIG. 13, optional step 5 can be carried out (by repeating steps 1-4) one or more times to accomplish multiple deliveries of the DNA-based vaccine in accordance with a “prime-prime” vaccination protocol, as discussed above.

FIG. 13b illustrates another example of this optional feature of this invention. The process illustrated in FIG. 13b is essentially the same as the process illustrated in FIG. 13a, except that the process of FIG. 13 is a “prime-boost” vaccination protocol. Note, in this regard that the plasma assist technology of this invention is not applied to the “boost” sites of this protocol, i.e., sites where the protein encoded by the DNA-based vaccine has been injected, since this injected protein is not intended to be delivered to inside the target cells of interest.

In carrying out this optional embodiment of this invention, any type of cold plasma can be used to carry out step 1 before the DNA-based vaccine is topically applied. Desirably, however, the same cold plasma system and operation are used to carry out this plasma step as are used in step 4 to carry out the plasma assist technology of this invention, as described above. That is to say, the same DBD cold plasma generators operated in the same way as described above in connection with this invention are used to carry out step 1.

### WORKING EXAMPLES

In order to more thoroughly describe this invention, the following working examples are provided.

### Example 1

A series of runs was conducted in which 8 week old female BALB/c mice were intradermally injected with 50 μl containing 40 μg of a DNA-based plasmid (Prime) encoding for HBsAg (Hepatitis B surface antigen) followed on day 14 with a second intradermal injection of an additional 50 μl containing 40 μg of the same DNA-based plasmid. This method will be referred to as the “prime-prime” method.

Immediately after each injection, a cold plasma was applied to the injection site using a planar large area DBD plasma generator with ambient air to generate the plasma. In some of these runs, the power source used was selected to deliver high voltage pulses having microsecond pulse widths, while in other runs the power source used was selected to deliver high voltage pulses having nanosecond pulse widths. The particular plasma conditions used are set forth in the following Table 1. Then on day 28, the mice were sacrificed, after which their spleens were recovered and analyzed for spot forming cells against the cytokine interferon gamma (IFN-γ). This was done by a conventional ELISpot assay that was run on three separate replicates taken from each spleen. Higher number of spot forming cells indicates a higher immune response.

Two control runs were also done in which no plasma was applied. In the first of these control runs, no DNA-based plasmid injection was made. In the second of these control runs, two DNA-based plasmid injections spaced 14 days apart were made in the same way as carried out in Groups 3-8.

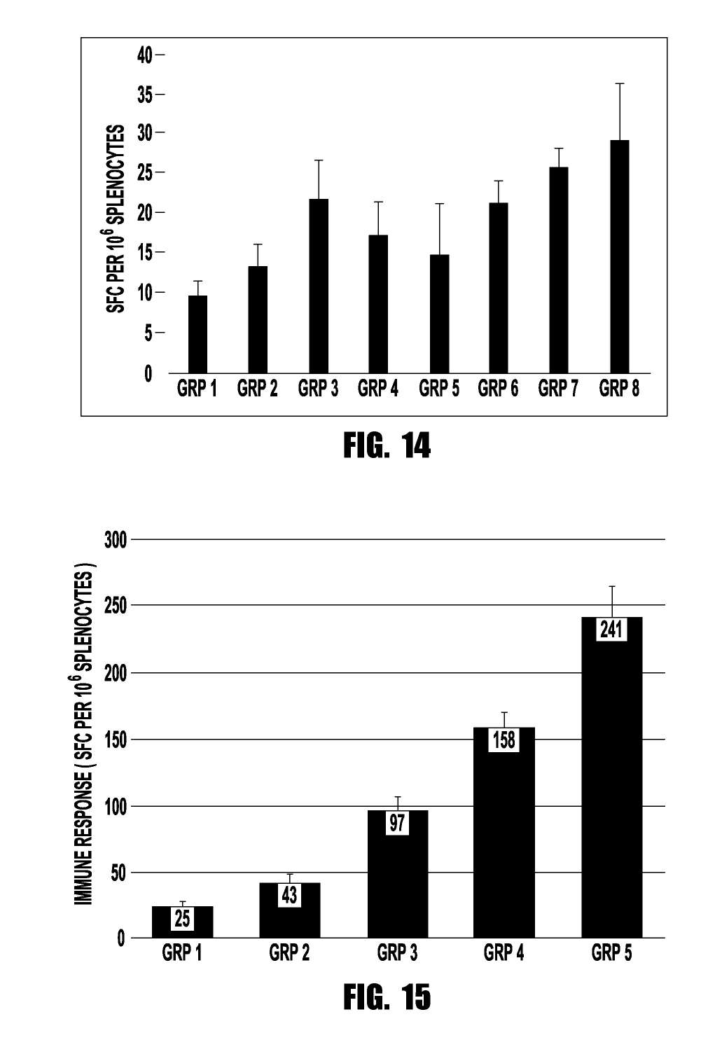

Eight different groups of mice were tested, each group containing three mice. The particular conditions to which each group of mice were subjected are set forth in the following Table 1, while the results obtained are graphically provided in FIG. 14.

TABLE 1
Test Conditions

| Group | Plasmid 1 | Plasmid 2 | Plasma Conditions |
|---|---|---|---|
| 1 | No | No | No plasma treatment |
| 2 | Yes | Yes | No plasma treatment |
| 3 | Yes | Yes | 16 kV, 5 μs, 2500 Hz, 30 seconds |
| 4 | Yes | Yes | 16 kV, 5 μs, 2500 Hz, 30 seconds |
| | | | followed 1 minute later by |
| | | | 16 kV, 5 μs, 2500 Hz, 30 seconds |
| 5 | Yes | Yes | 16 kV, 10 μs, 2500 Hz, 30 seconds |
| 6 | Yes | Yes | 20 kV, 260 ns, 500 Hz, 60 seconds |
| 7 | Yes | Yes | 20 kV, 260 ns, 25 pulses, |
| | | | followed 1 minute later by |
| | | | 20 kV, 260 ns, 20 pulses |
| 8 | Yes | Yes | 20 kV, 260 ns, 25 pulses |

As shown in FIG. 14, the effect of the plasma treatment of this invention was to improve the immune response of the mice subjected to this treatment by amounts ranging from 38 to 207% relative to Group 1, the control experiment in which the mice were neither injected with plasmids nor subjected to the plasma treatment and 10 to 122% relative to Group 2, the control experiment in which the mice were injected with the same amounts of plasmids but not subjected to the plasma treatment of this invention.

Note, also, that Group 8, which involved a single plasma treatment involving 20 20 kV pulses having a pulse width of 260 ns surprisingly showed the best enhancement in immune response compared to control Group 2, in which no get plasma treatment was applied.

### Example 2

Example 1 was repeated, except that in this case, on day 0 the mice were injected with 20 μg of the same DNA-based plasmid (Prime 1), while on day 14 the mice were injected 263 ng of the of the protein itself, i.e., HBsAg protein (Boost 1). This method will be referred to as the “prime-boost” method.

Five different groups of mice were tested, each group containing three mice. In the same way as in Example 1, two control runs were also carried out in which no plasma treatment was used. In the first of these control runs, no DNA-based plasmid nor protein was injected, while in the second of these control runs both the DNA-based plasmid and the protein were injected.

The particular plasma conditions to which each group of mice were subjected are set forth in the following Table 2. Meanwhile, the results (immune response) obtained, which are reported as Spot Forming Cells per 10⁶ Splenocytes, are graphically provided in FIG. 15.

TABLE 2
Test Conditions

| Group | Plasmid | Protein | Plasma Conditions |
|---|---|---|---|
| 1 | No | No | No plasma treatment |
| 2 | Yes | Yes | No plasma treatment |
| 3 | Yes | Yes | 20 kV, 500 ns, 25 pulses |
| 4 | Yes | Yes | 20 kV, 100 ns, 10 pulses, |
| | | | followed 1 minute later by |
| | | | 12 kV, 500 ns, 25 pulses |
| 5 | Yes | Yes | 17 kV, 5 μs, 2500 Hz, 30 seconds |

As shown in FIG. 15, the immune response exhibited by the mice in Group 5, which had been subjected to a 30 second plasma treatment with the a frequency of 2500 Hz, pulse duration of 5 μs at an applied pulsed voltage of 17 kV was some 9.6 (241/25) times or 865% better than that exhibited by the mice of Group 1, which had received no injections or plasma treatment. In addition, the immune response exhibited by the mice in Group 5 was some 5.6 (241/43) times or 467% greater than that exhibited by the mice of Group 2 which had received the same plasmid and protein injections but no plasma treatment.

Similarly, the immune response exhibited by the mice in Group 4, which had been subjected to two successive pulsed 500 nanosecond plasma treatments, was some 6.3 (158/25) times or 531% better than that exhibited by the mice of Group 1, which had received no injections or plasma treatment, and some 3.7 (158/43) times or 272% greater than that exhibited by the mice of Group 2 which had received the same plasmid and protein injections but no plasma treatment.

Finally, the immune response exhibited by the mice in Group 3, which had been subjected to a single pulsed 500 nanosecond plasma treatment, was some 3.9 (97/25) times or 286% better than that exhibited by the mice of Group 1, which had received no injections or plasma treatment, and 2.3 (97/43) times or 127% greater than that exhibited by the mice of Group 2 which had received the same plasmid and protein injections but no plasma treatment.

The results of these experiments demonstrate that the efficacy of a DNA-based vaccine in terms of eliciting a desired immune response can be substantially increased by the plasma-assist technology of this disclosure. Moreover, although these results are not directly comparable with those reported in the Connolly et publications mentioned above, nonetheless they do show that the inventive plasma-assist technology does provide a number of benefits relative to the processes described there.

For example, the full benefit of the plasma-assist technology of the Connolly et al. publications occurred only after 91 days had elapsed and 5 separate injections were made. In contract, the substantial benefit provided by the plasma-assist technology of this disclosure, as shown by the above working examples, occurred in only 28 days using only 2 injections. This suggests that the inventive plasma-assist technology should be able of providing its desired results better and faster than this earlier work.

Similarly, the above results further show that the inventive plasma-assist technology offers a greater degree of flexibility relative to other similar technologies in terms of customizing a particular plasma regimen to a particular DNA-based vaccine to be delivered, because of the numerous different variables that are available for affecting plasma application including applied voltage, pulse length, frequency, time of treatment, number of applied pulses and duty cycle, for example. This high degree of flexibility is not possible with plasma-assist technologies based on using DC current for plasma generation due to the greater degree of care that must be taken to prevent patient discomfort.

Although only a few embodiments of this invention have been described above, it should be appreciated that many modifications can be made without departing from the spirit and scope of the invention. All such modifications are intended to be included within the scope of this invention, which is to be limited only by the following claims:

1. A process for enhancing the efficacy of a DNA-based vaccine in connection with eliciting a desired immune response in which the DNA-based vaccine has been previously introduced into the body of a patient, the process comprising directing a non-thermal plasma at the application site where the DNA-based vaccine was introduced, wherein the non-thermal plasma is generated by a dielectric barrier discharge (DBD) plasma generator.

2. The process of claim 1, wherein the DBD plasma generator is a large area DBD plasma generator which is capable of generating a direct plasma essentially uniformly over an area of at least 5 cm².

3. The process of claim 2, wherein the DBD plasma generator is a jet-type plasma generator which is structured to convert a stream of a flowing gas into a jet of a direct plasma.

4. The process of claim 3, wherein the gas is helium.

5. The process of claim 1, wherein the DBD plasma generator generates a plasma in a gas, wherein the DBD plasma generator includes a high voltage electrode which is electrically insulated from contact with the gas, and further wherein the DBD plasma generator is powered by a power source which provides a high voltage potential drop between the high voltage electrode and ground, wherein the power source provides the high applied voltage in the form of voltage pulses having microsecond pulse widths of 1-50 μs, at applied voltages from 3 to 40 kV, at frequencies of 50 Hz to 5 kHz and duty cycles of 1-100%.

6. The process of claim 5, wherein the voltage pulses have pulse widths of 3-10 μs, at applied voltages of from 3 to 30 kV and frequencies of 1 kHz to 3.5 kHz.

7. The process of claim 5, wherein the non-thermal plasma is directed at the application site where the DNA-based vaccine was introduced by means of a plasma regimen involving one or more parts, and further wherein the total treatment time for each part of the plasma regimen lasts no longer than 120 seconds.

8. The process of claim 7, wherein the total treatment time lasts no longer than 90 seconds.

9. The process of claim 1, wherein the DBD plasma generator generates a plasma in a gas, wherein the DBD plasma generator includes a high voltage electrode which is electrically insulated from contact with the gas, and further wherein the DBD plasma generator is powered by a power source which provides a high voltage potential drop between the high voltage electrode and ground, wherein the power source provides the high applied voltage in the form of voltage pulses having nanosecond pulse widths of 1 and 999 ns at applied voltages of from 2 to 40 kV.

10. The process of claim 9, wherein the applied voltage is from 10-28 kV and the pulse width is from 75 to 600 ns.

11. The process of claim 9, wherein actuation of the power source to provide individual discrete pulses is done manually, wherein the non-thermal plasma is applied by means of a plasma regimen involving one or more parts, and further wherein the total number of voltage pulses applied in all parts combined is no more than 100.

12. The process of claim 10, in which the total number of voltage pulses applied in all parts combined is no more than 50.

13. The process of claim 9, wherein the non-thermal plasma is applied to the application site where the DNA-based vaccine was introduced by means of a plasma regimen involving a first part and a second part, wherein the total number of voltage pulses in the first part does not exceed 50, the total number of voltage pulses in second part also does not exceed 50 and the total number of voltage pulses in both the first and second parts combined does not exceed 100.

14. The process of claim 9, wherein the power source generates voltage pulses automatically at frequencies of 200 Hz to 1 kHz and further wherein the total time over which the plasma treatment occurs is no more than 120 seconds.

15. The process of claim 1, wherein the DBD plasma generator generates a plasma in a gas, wherein the DBD plasma generator includes a high voltage electrode which is electrically insulated from contact with the gas, and further wherein the DBD plasma generator is powered by a power source which provides a high voltage potential drop between the high voltage electrode and ground, wherein the power source provides the high applied voltage in the form of in the four of a simple alternating voltage wave form whose amplitude ranges from 2 to 40 kV, peak to peak at frequencies of 1 to 30 kHz.

16. The process of claim 1, wherein the patient is electrically connected to ground.

17. The process of claim 1, wherein the DBD cold plasma generator further includes a counter electrode, which is connected to ground.

18. The process of claim 1, wherein the DNA-based vaccine encodes an immunogenic antigen, wherein the DNA-based vaccine is applied to the patient by means of a vaccination protocol, wherein the vaccination protocol includes a first delivery inside the patient's body of the DNA-based vaccine, and further wherein the vaccination protocol also includes one or more subsequent deliveries in which the substance being delivered in each subsequent delivery is independently selected from either another dose of the DNA-based vaccine or the immunogenic antigen encoded by the DNA-based vaccine.

19. The process of claim 18, wherein a non-thermal plasma generated by a DBD plasma generator is directed at the application site on the skin of the patient's body where the first delivery of DNA-based vaccine inside the body was made.

20. The process of claim 19, wherein another dose of the DNA-based vaccine is made in one or more subsequent deliveries, and further wherein a non-thermal plasma generated by a DBD plasma generator is directed at the application site on the skin of the patient's body where at least one of these subsequent deliveries of DNA-based vaccine inside the body was made.

21. The process of claim 20, wherein a non-thermal plasma generated by a DBD plasma generator is directed at the respective application sites on the skin of the patient's body where each of these subsequent deliveries of DNA-based vaccine inside the body was made.

22. The process of claim 20, wherein each application of non-thermal plasma is done by means of a plasma regimen having two parts, wherein the voltage difference applied in the second part is at least 5 kV less than the voltage difference applied in the first part and further wherein the pulse width of the voltage pulses in the second part is at least twice as long as the pulse width of the voltage pulses in the first part.

23. The process of claim 20, wherein each application of non-thermal plasma is done by means of a plasma regimen having two parts, wherein the voltage difference applied in the second part is at least 5 kV more than the voltage difference applied in the first part and further wherein the pulse width of the voltage pulses in the second part is half as short or less as the pulse width of the voltage pulses in the first part.

24. The process of claim 18, wherein each delivery is independently spaced from the preceding delivery by a period of time ranging from 10 to 40 days.

25. The process of claim 24, wherein the period of time is from 14 to 28 days.

26. The process of claim 1, wherein the non-thermal plasma is applied by means of a plasma regimen composed of a first part and a second part, and further wherein the voltage difference applied in the second part is at least 5 kV less than the voltage difference applied in the first part and further wherein the pulse width of the voltage pulses in the second part is at least twice as long as the pulse width of the voltage pulses in the first part.

27. The process of claim 26, wherein the pulse widths of the voltage pulses in both the first part and the second part are each independently 1-50 μs.

28. The process of claim 26, wherein the pulse widths of the voltage pulses in both the first part and the second part are each independently 1-999 ns.

29. The process of claim 26, wherein the pulse width of the voltage pulses in the first part is 1-50 μs and the pulse width of the voltage pulses in the second part is 1-999 ns.

30. The process of claim 26, wherein the pulse width of the voltage pulses in the first part is 1-999 ns and the pulse width of the voltage pulses in the second part is 1-50 μs.

31. The process of claim 1, wherein the non-thermal plasma applied after at least one delivery of DNA-based vaccine is made by a plasma regimen composed of a first part and a second part, and further wherein the voltage difference applied in the second part is at least 5 kV more than the voltage difference applied in the first part and further wherein the pulse width of the voltage pulses in the second part is one-half or less as long as the pulse width of the voltage pulses in the first part.

32. The process of claim 31, wherein the pulse widths of the voltage pulses in both the first part and the second part are each independently 1-50 μs.

33. The process of claim 31, wherein the pulse widths of the voltage pulses in both the first part and the second part are each independently 1-999 ns.

34. The process of claim 31, wherein the pulse width of the voltage pulses in the first part is 1-50 μs and the pulse width of the voltage pulses in the second part is 1-999 ns.

35. The process of claim 31, wherein the pulse width of the voltage pulses in the first part is 20 and 999 ns and the pulse width of the voltage pulses in the second part is 1-50 μs.

36. The process of claim 1, wherein the DNA-based vaccine is introduced into the body of a patient from a location outside the patient's body by a transdermal delivery process comprising applying a non-thermal plasma to the skin of the patient and thereafter topically applying the DNA-based vaccine to the surface of the skin of the patient where this non-thermal plasma was applied, thereby allowing the DNA-based vaccine to migrate to inside the patient's body.

37. The process of claim 36, wherein after the DNA-based vaccine is topically applied to the surface of the patient's skin and before a non-thermal plasma is directed at this application site, a time delay of at least 1 minute occurs to enable the DNA-based vaccine to migrate into the patient's body.

38. The process of claim 1, wherein the non-thermal plasma is at atmospheric pressure.

39. The process of claim 1, wherein an electric field of 50-300 kV/cm is generated on the application site where the DNA-based vaccine was introduced.

40. The process of claim 1, wherein the DBD plasma generator is powered by a power source which provides an applied voltage in the form of voltage pulses, and further wherein the pulse width of the voltage pulses is 1-100 ns and the frequency of the pulses is 1-20 kHz.

41. The process of claim 20, wherein the DBD plasma generator is powered by a power source which provides an applied voltage in the form of voltage pulses, and further wherein the pulse width of the voltage pulses is 100-999 ns and the frequency of the pulses is 1 Hz-1 kHz.
